(19) United States
(12) Patent Application Publication
(10) Pub. No.: US 2015/0343167 A1
(43) Pub. Date: Dec. 3, 2015

(54) SUBJECT INTERFACE APPLIANCE HEATING SYSTEM

(71) Applicant: KONINKLIJKE PHILIPS N.V., Eindhoven (NL)

(72) Inventor: ROBERT GERARD RYBICKI, PITSBURGH, PA (US)

(21) Appl. No.: 14/760,748

(22) PCT Filed: Jan. 2, 2014
(86) PCT No.: PCT/IB2014/058012

§ 371 Date: Jul. 14, 2015

Related U.S. Application Data
(60) Provisional application No. 61/752,560, filed on Jan. 15, 2013.

(51) Int. Cl. A61M 16/10; A61M 16/16; A61M 16/08; A61M 16/00; A61M 16/06

(52) U.S. Cl. CPC A61M 16/1095; A61M 16/0003; A61M 16/0057; A61M 16/06; A61M 16/0875; A61M 16/161; A61M 2205/3368; A61M 2205/8237; A61M 2205/36

(57) ABSTRACT

Systems and methods for heating a flow of breathable gas during delivery to a subject use a subject interface appliance (184) that carries an interface heating system (160). The subject interface appliance further comprises an interface body (185) and a rechargeable power source (170). For respiratory therapy that includes the use of a humidifier, condensation or rainout may be reduced by virtue of heating the subject interface appliance.

### CROSS-REFERENCE TO RELATED APPLICATIONS

This patent application claims the priority benefit under 35 U.S.C. §119(e) of U.S. Provisional Application No. 61/752,560 filed on Jan. 15, 2013, the contents of which are herein incorporated by reference.

### BACKGROUND OF THE INVENTION

1. Field of the Invention

The present disclosure pertains to systems and methods for providing respiratory therapy of a subject. In particular, the present disclosure pertains to reducing and/or inhibiting condensation or rainout during therapy by heating the subject interface appliance.

2. Description of the Related Art

Some types of respiratory therapy involve the delivery of a pressurized flow of breathable gas to the airway of a subject. A therapy session may (be intended to) span eight or more hours, and may (be intended to) coincide and/or overlap, at least in part, with a subject's daily and/or nightly sleeping period. A subject's comfort during a therapy session is a useful factor in therapy adoption rates and/or therapy success rates. Humidification of the pressurized flow of breathable gas may improve a subject's comfort. Heated humidification may improve a subject's comfort. Condensation may form along the subject interface of respiratory therapy systems that include heated humidification. The formation of condensation, or rainout, has various downsides, including but not limited to reduced comfort of the subject and/or reduced therapy success rates.

### SUMMARY OF THE INVENTION

Accordingly, one or more embodiments of the present disclosure provide a subject interface appliance configured to be installed on the face of a subject to deliver a pressurized flow of breathable gas to the airway of the subject. The subject interface appliance comprises an interface body, a power source, and an interface heating system. The interface body is configured to be removably coupled to a heated or non-heated conduit of a respiratory circuit. The interface body is further configured to be installed on the face of the subject to place the airway of the subject in fluid communication with the conduit for delivery of a pressurized flow of breathable gas through the conduit to the airway of the subject. The interface body is further configured to form a cavity between the interface body and the face of the subject. The power source is carried by the interface body. The power source may be rechargeable. The power source may be configured to be recharged, e.g. through inductive charging. The interface heating system is carried by the interface body, the interface heating system being configured to controllably heat the cavity. The interface heating system is configured to be powered by the power source.

It is yet another aspect of one or more embodiments of the present disclosure to provide a method to heat a flow of breathable gas during delivery to the airway of a subject. The method is implemented using a subject interface appliance configured to be installed on the face of the subject. The method comprises installing an interface body on the face of a subject, the interface body being removably coupled to a conduit of a respiratory circuit, such that the installing step places the airway of the subject in fluid communication with the conduit for delivery of a flow of breathable gas through the conduit to the airway of the subject, and such that the installing step forms a cavity between the interface body and the face of the subject; carrying, by the interface body, a power source, the power source being configured to be recharged, e.g. through inductive charging; carrying, by the interface body, an interface heating system, the interface heating system being configured to be powered by the power source; and controllably heating, by the interface heating system, the cavity. This may allow for an increased amount of water vapor to be delivered to the subject before rain out on the interface body occurs.

It is yet another aspect of one or more embodiments to provide a subject interface appliance configured for heating a flow of breathable gas during delivery to the airway of a subject. The subject interface appliance comprises means for forming a cavity between the interface body and the face of the subject by installation on the face of a subject, the means being removably coupled to a conduit of a respiratory circuit, wherein the means is configured to place the airway of the subject in fluid communication with the conduit for delivery of a flow of breathable gas through the conduit to the airway of the subject; means for carrying a power source, the power source being configured to be recharged, e.g., through inductive charging; means for carrying an interface heating system, the interface heating system being configured to be powered by the power source; and means for controllably heating the cavity.

These and other aspects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 schematically illustrates a system configured to heat a subject interface appliance, according to certain embodiments;

FIG. 2 illustrates a subject interface appliance, according to certain embodiments; and

FIG. 3 illustrates a method for heating a subject interface appliance, according to certain embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled to move as one while maintaining a constant orientation relative to each other.

As used herein, the word “unitary” means a component is created as a single piece or unit. That is, a component that includes pieces that are created separately and then coupled as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

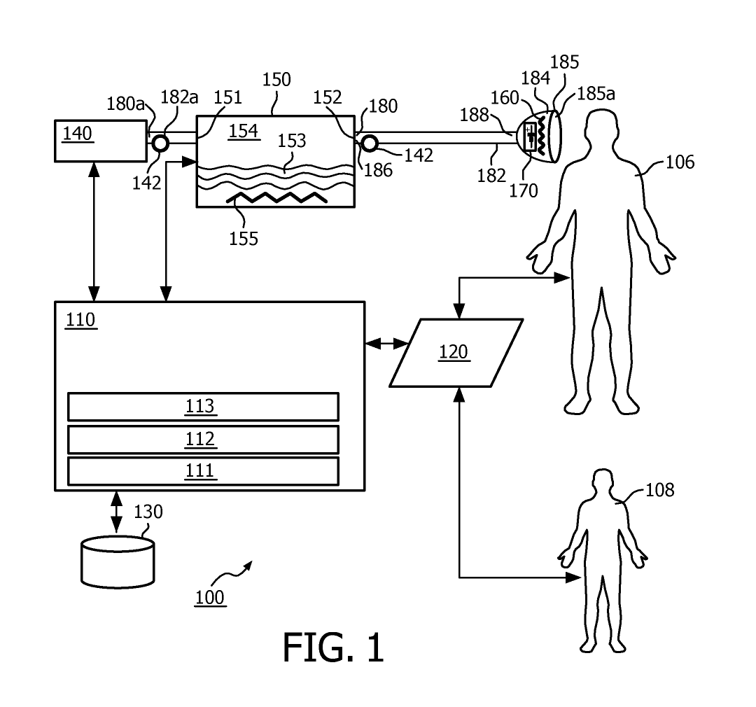

FIG. 1 schematically illustrates a system 100 configured to heat a subject interface appliance and/or heat a pressurized flow of humidified breathable gas during delivery to the airway of a subject 106. System 100 may be implemented as, integrated with, and/or operating in conjunction with a respiratory device that provides a flow of breathable gas along a flow path to subject 106. Humidifying the flow of gas, e.g. by adding moisture in the form of water vapor, may improve the experience and/or comfort of subject 106. Humidification may be beneficial for other reasons than those stated herein, as used in relevant fields of technology. The amount and/or level of moisture added, which may e.g. be expressed as a relative humidity percentage, may be monitored and/or controlled, e.g. through feedback, within system 100.

System 100 may include one or more of a pressure generator 140, a humidifier 150, a subject interface 180, an interface heating system 160, a power source 170, one or more sensors 142, an electronic storage 130, a user interface 120, a processor 110, a parameter determination module 111, a control module 112, a temperature module 113, and/or other components. System 100 may be configured to provide respiratory therapy to subject 106.

Pressure generator 140 of system 100 in FIG. 1 may be integrated, combined, or connected with a ventilator and/or (positive) airway pressure device (PAP/CPAP/BiPAP®/etc.) and configured to provide a pressurized flow of breathable gas for delivery to the airway of subject 106, e.g. via one or more subject interfaces 180. Subject interface 180 may sometimes be referred to as a delivery circuit.

Pressure generator 140 may be integrated, combined, or connected with humidifier 150 in a configuration that is configured to humidify a flow of breathable gas within system 100. In the configuration depicted in FIG. 1, pressure generator 140 fluidly communicates with humidifier 150 via a subject interface 180a, which may be structurally and/or functionally similar to subject interface 180, at least in part. As depicted in FIG. 1, humidifier 150 fluidly communicates, via subject interface 180, with the airway of subject 106, after the flow of breathable gas passes interface heating system 160. The configuration of various components in FIG. 1 is not intended to limit the scope of the described technology in any way. For example, in some embodiments, interface heating system 160 may be disposed between pressure generator 140 and humidifier 150. In some embodiments, humidifier 150 may be disposed upstream from pressure generator 140.

Respiratory therapy may be implemented as pressure control, pressure support, volume control, and/or other types of support and/or control. For example, to support inspiration, the pressure of the pressurized flow of breathable gas may be adjusted to an inspiratory pressure. Alternatively, and/or simultaneously, to support expiration, the pressure and/or flow of the pressurized flow of breathable gas may be adjusted to an expiratory pressure. Other schemes for providing respiratory support and/or ventilation through the delivery of the pressurized flow of breathable gas are contemplated. Subject 106 may but need not initiate one or more phases of respiration.

System 100 may be configured to adjust and/or maintain levels of pressure, flow, humidity, velocity, acceleration, and/or other parameters of the humidified, pressurized flow of breathable gas. One or more adjustments may occur in substantial synchronization with the breathing cycle of the subject. In some embodiments, one or more operating levels (e.g. pressure, volume, etc.) are adjusted on a relatively ongoing manner (e.g., each breath, every few breaths, every few seconds, etc.) during an individual session of respiratory therapy to titrate the therapy. Alternatively, and/or simultaneously, adjustments to one or more operating levels of system 100 and/or any component thereof may be made more intermittently and/or between therapy sessions rather than during a particular therapy session.

A pressurized flow of breathable gas may be delivered from pressure generator 140 to the airway of subject 106 via one or more subject interfaces 180. Subject interface 180 may include a conduit 182 and/or a subject interface appliance 184. As depicted in FIG. 1, subject interface 180a may include a conduit 182a. Subject interface 180 may include conduit 182. Conduit 182 and/or conduit 182a may include a flexible length of hose, or other conduit. As depicted in FIG. 1, conduit 182 may place subject interface appliance 184 in fluid communication with humidifier 150, and, indirectly, with pressure generator 140. As depicted in FIG. 1, subject interface 180 may include a proximal end 186 disposed at or near humidifier 150 and a distal end 188 disposed at or near subject interface appliance 184. Conduit 182 and/or conduit 182a form a flow path through which the pressurized flow of breathable gas is communicated between subject interface appliance 184, humidifier 150, and pressure generator 140.

Subject interface appliance 184 of system 100 in FIG. 1 is configured to deliver the pressurized flow of breathable gas to subject 106, e.g. to the airway of subject 106. Subject interface appliance 184 may be configured to reduce and/or inhibit condensation from forming along the path of delivery of a (humidified and/or pressurized) flow of breathable gas to subject 106. Subject interface appliance 184 may include an interface body 185, an interface heating system 160, a power source 170, and any appliance suitable for the described function.

In one embodiment, pressure generator 140 is a dedicated ventilation device and subject interface appliance 184 is configured to be removably coupled with another interface appliance being used to deliver respiratory therapy to subject 106. For example, subject interface appliance 184 may be configured to engage with and/or be inserted into an endotracheal tube, a tracheotomy portal, and/or other interface appliances. In one embodiment, subject interface appliance 184 is configured to engage the airway of subject 106 without an intervening appliance. In this embodiment, subject interface appliance 184 may include one or more of an endotracheal tube, a nasal cannula, a tracheotomy tube, a nasal mask, a nasal/oral mask, a full-face mask, a total facemask, and/or other interface appliances that communicate a flow of gas with an airway of a subject. The present disclosure is not limited to these examples, and contemplates delivery of the pressurized flow of breathable gas to subject 106 using any subject interface. In some embodiments, interface heating system 160 may be embedded and/or integrated within subject interface appliance 184.

Interface body 185 of subject interface appliance 184 may be configured to be removably coupled to conduit 182. Interface body 185 may be configured to be installed in the face of subject 106 to place the airway of subject 106 in fluid communication with conduit 182 for delivery of a (humidified and/or pressurized) flow of breathable gas through conduit 182 to the airway of subject 106. Interface body 185 may be configured to form a cavity 185a between interface body 185 and the face of subject 106. For example, cavity 185a may be formed responsive to installation of subject interface appliance 184 on the face of subject 106.

Power source 170 may be carried by interface body 185. Power source 170 may include a rechargeable power source. In some embodiments, power source 170 may include a primary cell. Power source 170 may be implemented by a battery. For example, power source 170 may include moldable materials such as may be used in a lithium ion gel battery. Power source 170 may be molded to fit within interface body 185, subject interface appliance 184, and/or a combination thereof. For example, power source 170 may be integrated, embedded, combined, and/or sealed within subject interface appliance 184 such that subject 106 is not exposed to power source 170, and vice versa. In other words, conduction of current (e.g. originating from power source 170) between subject interface appliance 184 and subject 106 is prevented by virtue of power source 170 being integrated, embedded, combined, and/or sealed within subject interface appliance 184. Power source 170 may be configured to be recharged through hard contact charging, inductive charging, and/or other techniques of charging.

Interface heating system 160 may be carried by interface body 185. Interface heating system 160 of subject interface appliance 184 in FIG. 1 is configured to controllably heat cavity 185a and/or a flow of breathable gas within subject interface appliance 184, particularly within subject interface 180. As depicted in FIG. 1, interface heating system 160 is configured to heat the humidified, pressurized flow of breathable gas within subject interface 180 en route to the airway of subject 106. The depiction of interface heating system 160 as a single component is not intended to be limiting in any way. In some embodiments, interface heating system 160 may be configured to be powered by power source 170.

Interface heating system 160 may be implemented as a heated coil wrapped around and/or embedded within interface body 185 and/or other components of interface 180. Other ways to add heat to system 100 are contemplated, as used in relevant fields of technology. Thermal exchange through interface heating system 160 is not limited to an embodiment using one or more heated coils. In some embodiments, interface heating system 160 may be embedded and/or integrated within another component of subject interface appliance 184. For example, power source 170 and interface heating system 160 may be integrated, embedded, combined, and/or sealed within subject interface appliance 184 such that maintenance and/or cleaning of subject interface appliance 184 does not expose interface heating system 160 nor power source 170, e.g. to ambient atmosphere, moisture/liquid, etc.

In other words, by sealing power source 170 and interface heating system 160 within subject interface appliance 184, subject 106 or another user may wash subject interface appliance 184 without harming any components, including electric and/or conductive components. In some embodiments, power source 170 and interface heating system 160 may be sealed within subject interface appliance 184 such that subject interface appliance 184 is one or more of waterproof, water-resistant, resistance to the ingress of water and/or water-vapor, splash-proof, condensation-proof, shock-proof, and/or in any other way capable to withstand external influences or conditions of use without sustaining damage to power source 170 or interface heating system 160.

Humidifier 150 may be configured to controllably humidify the flow of breathable gas in system 100. In some embodiments, humidifier 150 is configured to controllably heat a liquid such that vapor formed from the heated liquid adds moisture to a flow of breathable gas. Humidifier 150 may include one or more of a gas inlet 151, a gas outlet 152, a humidification chamber 154 configured to contain liquid, liquid 153 intended to be vaporized, a heating element 155, and/or other components. A flow of breathable gas may be received through gas inlet 151. The flow of breathable gas may be humidified within humidification chamber 154 by liquid vapor formed from liquid 153 being heated. Liquid 153 may be heated through heating element 155. The humidified flow of breathable gas may be released from humidification chamber 154 and/or humidifier 150 through gas outlet 152. In some embodiments, heating element 155 may be disposed at or near the bottom of humidification chamber 154 and/or in proximity to liquid 153 within humidification chamber 154. In some embodiments, heating element 155 may not come in direct contact with liquid 153. The heat emitted by heating element 155 may be dispensed into liquid 153, e.g. indirectly, and vaporize liquid 153.

As depicted in FIG. 1, gas inlet 151 may fluidly communicate with subject interface 180a to receive a flow of pressurized breathable gas from pressure generator 140, disposed upstream. As depicted in FIG. 1, gas outlet 152 may fluidly communicate with subject interface 180 to guide the humidified, pressurized flow of breathable gas from humidifier 150 to the airway of subject 106 via subject interface appliance 184. Humidifier 150, as depicted in FIG. 1 is disposed downstream from subject interface appliance 184. In some embodiments, the humidified, pressurized flow of breathable gas may include medicament.

Humidity of air or other breathable gases varies with temperature. As a consequence, a temperature differential, e.g. anywhere along subject interface 180, may affect the relative humidity within subject interface 180. In particular, a temperature differential along subject interface 180 may cause condensation.

Electronic storage 130 of system 100 in FIG. 1 comprises electronic storage media that electronically stores information. The electronic storage media of electronic storage 130 may include one or both of system storage that is provided integrally (i.e., substantially non-removable) with system 100 and/or removable storage that is removably connectable to system 100 via, for example, a port (e.g., a USB port, a FireWire port, etc.) or a drive (e.g., a disk drive, etc.).

Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EPROM, EEPROM, RAM, etc.), solid-state storage media (e.g., flash drive, etc.), and/or other electronically readable storage media. Electronic storage 130 may store software algorithms, information determined by processor 110, information received via user interface 120, and/or other information that enables system 100 to function properly. For example, electronic storage 130 may record or store one or more gas and/or respiratory parameters (as discussed elsewhere herein), and/or other information. Electronic storage 130 may be a separate component within system 100, or electronic storage 130 may be provided integrally with one or more other components of system 100 (e.g., processor 110).

User interface 120 of system 100 in FIG. 1 is configured to provide an interface between system 100 and a user (e.g., user 108, subject 106, a caregiver, a therapy decision-maker, etc.) through which the user can provide information to and receive information from system 100. This enables data, results, and/or instructions and any other communicable items, collectively referred to as “information,” to be communicated between the user and system 100. An example of information that may be conveyed to user 108 is a report detailing operational settings of subject interface appliance 184 as selected and/or preferred by subject 106. An example of information that user 108 or subject 106 may provide to system 100 is a target temperature or target humidity level during respiratory therapy. Examples of interface devices suitable for inclusion in user interface 120 include a keypad, buttons, switches, a keyboard, knobs, dials, levers, a display screen, a touch screen, speakers, a microphone, an indicator light, an audible alarm, and a printer. Information may be provided to user 108 or subject 106 by user interface 120 in the form of auditory signals, visual signals, tactile signals, and/or other sensory signals.

It is to be understood that other communication techniques, either hard-wired or wireless, are also contemplated herein as user interface 120. For example, in one embodiment, user interface 120 may be integrated with a removable storage interface provided by electronic storage 130. In this example, information is loaded into system 100 from removable storage (e.g., a smart card, a flash drive, a removable disk, etc.) that enables the user(s) to customize the embodiment of system 100. Other exemplary input devices and techniques adapted for use with system 100 as user interface 120 include, but are not limited to, an RS-232 port, RF link, an IR link, modem (telephone, cable, Ethernet, internet or other). In short, any technique for communicating information with system 100 is contemplated as user interface 120.

One or more sensors 142 of system 100 in FIG. 1 are configured to generate output signals conveying measurements related to parameters of the flow of breathable gas within system 100. These parameters may include one or more of flow, (airway) pressure, barometric pressure, temperature, humidity, velocity, acceleration, and/or other parameters. One or more sensors 142 may be in fluid communication with conduit 182a, conduit 182, subject interface appliance 184, and/or other components of system 100. One or more sensors 142 may generate output signals related to physiological parameters pertaining to subject 106.

One or more sensors 142 may generate output signals conveying information related to parameters associated with the state and/or condition of an airway of subject 106, the breathing of subject 106, the breathing rate of subject 106, the gas delivered to subject 106, the composition, temperature, and/or humidity of the gas delivered to subject 106, the delivery of the gas to the airway of subject 106, and/or a respiratory effort by the subject. For example, a parameter may be related to a mechanical unit of measurement of a component of pressure generator 140 (or of a device that pressure generator 140 is integrated, combined, or connected with) such as valve drive current, rotor speed, motor speed, blower speed, fan speed, or a related measurement that may serve as a proxy for any of the previously listed parameters through a previously known and/or calibrated mathematical relationship. Resulting signals or information from one or more sensors 142 may be transmitted to processor 110, user interface 120, electronic storage 130, and/or other components of system 100. This transmission may be wired and/or wireless.

The illustration of sensor 142 including three members in FIG. 1 is not intended to be limiting. The illustration of a sensor 142 at or near subject interface appliance 184 is not intended to be limiting, though that location may be preferred in some embodiments to provide feedback and/or information regarding the current temperature and/or current humidity of the humidified, pressurized flow of breathable gas being delivered to the airway of subject 106. For example, this current temperature and/or current humidity may function as feedback for a target temperature or set point (and/or a target humidity level) for controlling interface heating system 160. Note that a temperature differential may occur along subject interface 180.

The illustration of the location of one or more sensors 142 in FIG. 1 is not intended to be limiting, though those locations may be preferred in some embodiments to provide feedback and/or information regarding the current temperature and/or humidity of the flow of breathable gas being delivered. For example, this current temperature may function as feedback for a target temperature.

Processor 110 of system 100 in FIG. 1 is configured to provide information processing capabilities in system 100. As such, processor 110 includes one or more of a digital processor, an analog processor, a digital circuit designed to process information, an analog circuit designed to process information, and/or other mechanisms for electronically processing information. Although processor 110 is shown in FIG. 1 as a single entity, this is for illustrative purposes only. In some embodiments, processor 110 includes a plurality of processing units.

As is shown in FIG. 1, processor 110 is configured to execute one or more computer program modules. The one or more computer program modules include one or more of parameter determination module 111, control module 112, temperature module 113, and/or other modules. Processor 110 may be configured to execute modules 111-113 by software; hardware; firmware; some combination of software, hardware, and/or firmware; and/or other mechanisms for configuring processing capabilities on processor 110.

It should be appreciated that although modules 111-113 are illustrated in FIG. 1 as being co-located within a single processing unit, in embodiments in which processor 110 includes multiple processing units, one or more of modules 111-113 may be located remotely from the other modules. The description of the functionality provided by the different modules 111-113 described herein is for illustrative purposes, and is not intended to be limiting, as any of modules 111-113 may provide more or less functionality than is described. For example, one or more of modules 111-113 may be eliminated, and some or all of its functionality may be provided by other ones of modules 111-113. Note that processor 110 may be configured to execute one or more additional modules that may perform some or all of the functionality attributed below to one of modules 111-113. In some embodiments, some or all of the described functionality of an individual computer program module may be incorporated, shared, embedded, and/or integrated into one or more other computer program modules or elsewhere within system 100.

In some embodiments, parameter determination module 111 may be configured to determine one or more gas parameters, breathing parameters, and/or other parameters from output signals generated by sensor(s) 142. The one or more gas parameter may include and/or be related to one or more of (peak) flow, flow rate, (tidal) volume, pressure, temperature, humidity, velocity, acceleration, gas composition (e.g. concentration(s) of one or more constituents such as, e.g., water vapor or CO₂), thermal energy dissipated, and/or other measurements related to the pressurized flow of breathable gas or the conditions within the cavity formed by interface body 185, e.g. during installation of subject interface appliance 184 on the face of subject 106. For example, one or more of these gas parameters, such as pressure and/or volume, may be used by the control module, described elsewhere herein, during respiratory therapy.

One or more breathing parameters may be derived, e.g. by parameter determination module 111, from gas parameters and/or from sensor-generated output signals conveying measurements of, e.g., the pressurized flow of breathable gas. The one or more breathing parameters may include one or more of respiratory rate, breathing period, inhalation time or period, exhalation time or period, respiration flow curve shape, transition time from inhalation to exhalation and/or vice versa, transition time from peak inhalation flow rate to peak exhalation flow rate and/or vice versa, respiration pressure curve shape, maximum proximal pressure drop (per breathing cycle and/or phase), and/or other breathing parameters. Note that the temperature and/or humidity within cavity 185a may change as a result of exhalation of subject 106.

In some embodiments, control module 112 is configured to control one or more of pressure generator 140, power source 170, interface heating system 160, and/or other components of system 100.

Control module 112 may be configured to control pressure generator 140 to adjust one or more gas parameters, described elsewhere herein, of the pressurized flow of breathable gas. Control may be in accordance with a respiratory therapy regimen, one or more algorithms that control adjustments and/or changes in the pressurized flow of breathable gas over time, operational settings, and/or other factors. For example, subject 106 or user 108 may provide one or more settings that correspond to one or more particular pressure levels, one or more modes of operation, and/or one or more preferences related to the operation of pressure generator 140. The control module may be configured to control pressure generator 140 to provide the pressurized flow of breathable gas. The control module may be configured to control pressure generator 140 such that one or more gas parameters of the pressurized flow of breathable gas are varied over time in accordance with a respiratory therapy regimen. The control module may be configured to control pressure generator 140 to provide the pressurized flow of breathable gas at inhalation pressure levels during inhalation phases, and/or at exhalation pressure levels during exhalation phases.

Control module 112 may be configured to control power source 170 to adjust operational settings and/or modes. In some embodiments, control module 112 may be configured to adjust the mode of operation of power source 170. For example, modes of operation may include a recharging mode, a mode in which power source 170 is turned off, a mode in which power source 170 supplies power to, by way of non-limiting example, interface heating system 160, and/or other modes of operation.

Control module 112 may be configured to control interface heating system 160 to adjust operational settings pertaining to heating cavity 185a, interface body 185, and/or any other component of subject interface appliance 184 or system 100. For example, control module 112 may be configured to maintain a target temperature and/or a target humidity for cavity 185a. Control module 112 may be configured to adjust operational settings to accomplish maintenance of a target temperature and/or a target humidity.

Parameters determined by other modules of system 100, received through sensors 142, and/or obtained through other ways may be used by control module 112, e.g. in a feedback manner, to adjust one or more operational settings and/or modes. Alternatively, and/or simultaneously, signals and/or information obtained through user interface 120 may be used by control module 112. Control module 112 may be configured to time its operations relative to transitional moments in the breathing cycle of a subject, over multiple breath cycles, and/or in any other relation to, e.g., any determinations by any of the computer program modules of system 100.

In some embodiments, temperature module 113 may be configured to determine a current temperature and/or a current humidity of a flow of breathable gas, cavity 185a, interface body 185, and/or another component of subject interface appliance 184. For example, the current temperature may pertain to the temperature of the humidified, pressurized flow of breathable gas within subject interface appliance 184. The current temperature and/or the current humidity may be determined based on one or more output signals generated by one or more sensors 142. For example, the current temperature may be determined based on a sensor 142 at or near subject interface appliance 184, as illustrated in FIG. 1. In some embodiments, temperature module 113 may be implemented as part of a parameter determination module, for example the parameter determination module described above.

Temperature module 113 may be configured to determine and/or obtain a target temperature and/or a target humidity for the flow of breathable gas, cavity 185a, interface body 185, and/or another component of subject interface appliance 184. For example, the target temperature may pertain to the temperature of the humidified, pressurized flow of breathable gas within subject interface appliance 184. In some embodiments, subject 106 and/or user 108 may provide and/or select the target temperature and/or the target humidity, for example through user interface 120. For example, subject 106 may adjust the target temperature based on personal preference.

In some embodiments, subject 106 may select a preferred offset temperature as the target temperature, wherein the offset temperature may be based on and/or relative to the current ambient temperature. In some embodiments, subject 106 may select a preferred offset humidity level as the target humidity level, wherein the offset may be based on and/or relative to the current ambient humidity level. Alternatively, and/or simultaneously, the target temperature and/or the target humidity level may be derived and/or determined based on one or more other selectable and/or adjustable operational settings. Target temperature and/or target humidity level may be configured to cooperate with control module 111 such that the target temperature and/or target humidity level of, for example, cavity 185a is maintained. Maintenance may be based on feedback from temperature and/or humidity measurements by one or more sensors 142.

In some embodiments, a controlled temperature and/or humidity level may change due to external influences, including but not limited to heat and/or moisture supplied by subject 106, heat and/or humidity lost due to mask leaks, heat and/or humidity lost due to (temporary) disengagement of subject interface appliance 184 from the face of subject 106, and/or other external influences. Control module 112 may be configured to attempt to counter occurrences of external influence on a controlled temperature. Control module 112 may be configured in such a way as to accomplish one or more of reducing a temperature differential along the flow path of delivered gas, improving comfort for subject 106, reducing or increasing the (relative) humidity by maintaining a target humidity for a changed temperature, and/or other adjustments, for example inhibiting and/or reducing condensation from forming along subject interface 180 or within subject interface appliance 184.

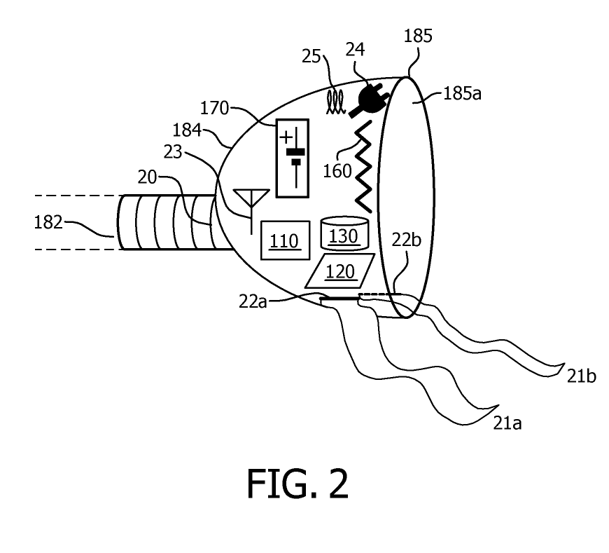

By way of illustration, FIG. 2 schematically illustrates an embodiment of subject interface appliance 184. As depicted, subject interface appliance 184 includes one or more of interface body 185, cavity 185a, power source 170, interface heating system 160, a conduit connector 20, one or more processors 110, electronic storage 130, user interface 120, a communication transceiver 23, a power plug 24, an inductive power receiver 25, mechanical connectors 22a and 22b, straps 21a and 21b, and/or other components. Interface body 185, cavity 185a, power source 170, and interface heating system 160 may operate in a manner similar to or the same as described above in relation to FIG. 1. Returning to FIG. 2, conduit connector 20 is configured to removably couple interface body 185 and/or subject interface appliance 184 to conduit 182 such that fluid communication may be established.

Communication transceiver 23 may be configured to establish communication with subject interface appliance 184 and/or any components included therein/thereon. For example, a subject may use user interface 120 to alter the flow of pressurized breathable gas coming from a pressure generator. Communication transceiver 23 may transfer and/or transmit instructions and/or commands, e.g. wirelessly, to cause operational settings of a pressure generator to be altered in accordance with instructions and/or commands from subject. In some embodiments, subject interface appliance 184 may operate in a coordinated manner with a humidifier such as humidifier 150 depicted in FIG. 1 such that the temperature of the humidified pressurized flow of breathable gas and the temperature within cavity 185a may be controlled to reduce and/or prevent condensation. Referring to FIG. 2, information may be received and/or transmitted by communication transceiver 23. Received and/or transmitted information may be processed by one or more processors 110.

Power plug 24 is configured to charge power source 170, e.g. through a conventional outlet. In some embodiments, power plug 24 may be removably connected to subject interface appliance 184. Inductive power receiver 25 is configured to charge power source 170 through inductive charging. Inductive power receiver 25 may be based on direct induction, electrodynamic induction, electrostatic induction, resonant magnetic induction, electromagnetic radiation, and/or other techniques for contactless energy transfer. For example, inductive power receiver 25 may include one or more (magnetic) metal coils. In some embodiments, inductive power receiver 25 may be embedded and/or sealed within subject interface appliance 184.

Power source may supply power to one or more components included in subject interface appliance 184 as depicted in FIG. 2. For example, power source 170 may be configured to supply power to communication transceiver 23 and/or any electronics included in subject interface appliance 184 to operate communication transceiver 23, including, but not limited to, user interface 120 and/or one or more processors 110. In some embodiments, a power source configured to supply power to inductively charge power source 170 through inductive power receiver 25 may be integrated, embedded, and/or combined with a pressure generator and/or a system such as system 100 depicted in FIG. 1.

Returning to FIG. 2, mechanical connectors 22a and 22b are configured to connect subject interface appliance 184, via e.g. straps 21a and 21b, to a structure that is suitable to keep subject interface appliance 184 in place on the face of a subject to form cavity 185a. Note that the number and location of mechanical connectors 22a and 22b and straps 21a and 21b as depicted in FIG. 1 is not intended to be limiting in any way. Embodiments may use more or less mechanical connectors and straps than depicted in FIG. 2. Straps 21a and 21b may connect to headgear, such as a helmet, so the subject can easily and comfortably wear subject interface appliance 184 in a proper position.

In some embodiments, mechanical connectors 22a and 22b may provide a communicative link and/or a power link to headgear. For example, such headgear may include an antenna that subject interface appliance 184 can use to establish communication with components of a system remote from subject interface appliance 184. For example, such headgear may include electronic components that serve a user interface function and that may be powered, through a power link, by power source 170. By way of non-limiting example, a user interface function served by electronic components included in headgear may be a set of lights that indicate the current temperature in cavity 185a. In some embodiments, one or more components depicted in FIG. 2 as being included in and/or carried by subject interface appliance 184 may, instead, be included in and/or carried by headgear and connected to subject interface appliance 184 through mechanical connectors 22a and 22b. In such embodiments, mechanical connectors 22a and 22b may be more aptly described as mechanical and electrical connectors 22a and 22b, or simply connectors 22a and 22b.

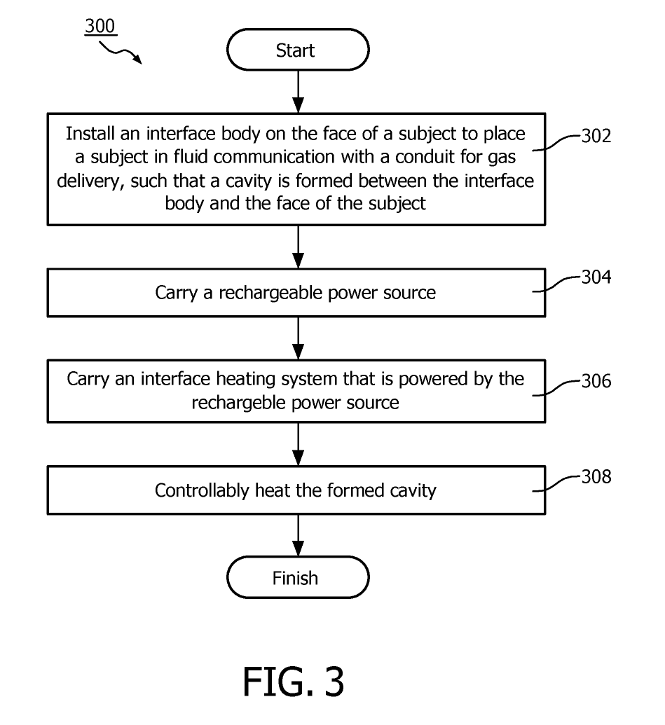

FIG. 3 illustrates a method 300 for heating a flow of breathable gas for delivery to a subject. The operations of method 300 presented below are intended to be illustrative. In certain embodiments, method 300 may be accomplished with one or more additional operations not described, and/or without one or more of the operations discussed. Additionally, the order in which the operations of method 300 are illustrated in FIG. 3 and described below is not intended to be limiting.

In certain embodiments, method 300 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 300 in response to instructions stored electronically on an electronic storage medium. The one or more processing devices may include one or more devices configured through hardware, firmware, and/or software to be specifically designed for execution of one or more of the operations of method 300.

At an operation 302, an interface body is installed on the face of a subject, the interface body being removably coupled to a conduit of a respiratory circuit, such that the installing step places the airway of the subject in fluid communication with the conduit for delivery of a flow of breathable gas through the conduit to the airway of the subject, and such that the installing step forms a cavity between the interface body and the face of the subject. In some embodiments, operation 302 is performed by an interface body the same as or similar to interface body 185 (shown in FIG. 1 and described herein).

At an operation 304, a power source is carried, the power source being configured to be recharged, e.g. through inductive charging. In some embodiments, operation 304 is performed by an interface body the same as or similar to interface body 185 (shown in FIG. 1 and described herein).

At an operation 306, an interface heating system is carried, the interface heating system being configured to be powered by the power source. In some embodiments, operation 306 is performed by an interface body the same as or similar to interface body 185 (shown in FIG. 1 and described herein).

At an operation 308 the cavity is heated in a controllable manner. Heating of the cavity may be controlled in a feedback manner based on a measured temperature in the cavity. In some embodiments, operation 308 is performed by an interface heating system the same as or similar to interface heating system 160 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the disclosure has been described in detail for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A subject interface appliance configured to be installed on the face of a subject to deliver a pressurized flow of breathable gas to the airway of the subject, the subject interface appliance comprising:
an interface body configured to be coupled to a conduit of a respiratory circuit, further configured to be installed on the face of the subject to place the airway of the subject in fluid communication with the conduit for delivery of a pressurized flow of breathable gas through the conduit to the airway of the subject, and further configured to form a cavity between the interface body and the face of the subject;
a power source carried by the interface body, the power source being configured to supply power and further configured to be recharged; and
an interface heating system carried by the interface body, the interface heating system being configured to controllably heat the cavity, wherein the interface heating system is configured to be powered by the power that is supplied by the power source.

2. The subject interface appliance of claim 1, wherein the power source is integrated within the subject interface appliance such that conduction of current between the subject interface appliance and the subject is prevented.

3. The subject interface appliance of claim 1, wherein the power source and the interface heating system are integrated within the subject interface appliance such that maintenance and/or cleaning of the subject interface appliance does not expose the interface heating system nor the power source.

4. The subject interface appliance of claim 1, further comprising one or more sensors configured to generate output signals conveying information related to a temperature and/or humidity within the cavity, wherein the interface heating system controllably heats the cavity based on the generated output signals.

5. The subject interface appliance of claim 1, wherein the power source is further configured to power a conduit heating system disposed in the conduit that heats the conduit.

6. A method to heating a flow of breathable gas during delivery to the airway of a subject, the method being implemented using a subject interface appliance includes an interface body configured to be installed on the face of the subject and further includes a power source and an interface heating system, the method comprising:
coupling the interface body removably to a conduit of a respiratory circuit, such that the coupling places the airway of the subject in fluid communication with the conduit for delivery of a flow of breathable gas through the conduit to the airway of the subject, wherein a cavity is formed between the interface body and the face of the subject;
carrying, by the interface body, a power source, the power source being configured to supply power and be recharged;
supplying, by the power source, the power to the interface heating system;
carrying, by the interface body, the interface heating system, the interface heating system being powered by the power that is supplied by the power source; and
controllably heating, by the interface heating system, the cavity.

7. The method of claim 6, the power source being integrated within the subject interface appliance such that conduction of current between the subject interface appliance and the subject is prevented.

8. The method of claim 6, further comprising:
integrating the power source and the interface heating system within the subject interface appliance such that maintenance and/or cleaning of the subject interface appliance does not expose the interface heating system nor the power source.

9. The method of claim 6, further comprising: generating output signals conveying information related to a temperature and/or humidity within the cavity, wherein controllably heating the cavity is based on the generated output signals.

10. The method of claim 6, further comprising:
supplying power, by the power source, to a conduit heating system to heat the conduit.

11. A subject interface appliance configured for heating a flow of breathable gas during delivery to the airway of a subject, the subject interface appliance comprising:
means for forming a cavity near the face of the subject by installation on the face of a subject, the means being removably coupled to a conduit of a respiratory circuit, wherein the means is configured to place the airway of the subject in fluid communication with the conduit for delivery of a flow of breathable gas through the conduit to the airway of the subject;
means for carrying a power source, the power source being configured to supply power and be recharged;
means for carrying an interface heating system, the interface heating system being powered by the power that is supplied by the power source; and
means for controllably heating the cavity.

12. The subject interface appliance of claim 11, the power source being integrated within the subject interface appliance such that conduction of current between the subject interface appliance and the subject is prevented.

13. The subject interface appliance of claim 11, wherein the power source and the interface heating system are integrated within the subject interface appliance such that maintenance and/or cleaning of the subject interface appliance does not expose the interface heating system nor the power source.

14. The subject interface appliance of claim 11, further comprising:
means for generating output signals conveying information related to a temperature and/or humidity within the cavity;
wherein operation of the means for controllably heating the cavity is based on the generated output signals.

15. The subject interface appliance of claim 11, wherein the power source is further configured to power a conduit heating system to heat the conduit.
